(19) United States
(12) Patent Application Publication
(10) Pub. No.: US 2026/0227373 A1
(43) Pub. Date: Aug. 6, 2026

(54) BIOSENSOR CHIP FOR THE DETECTION OF TOXINS IN FOOD PRODUCTS AND METHOD FOR FABRICATION THEREOF

(71) Applicants: BITS BioCyTiH FOUNDATION, Jhunjhunu (IN); Birla Institute of Technology & Science (BITS) Pilani, Jhunjhunu (IN)

(72) Inventors: Sunil BHAND, Zuarinagar (IN); Neelama M K, Zuarinagar (IN)

(21) Appl. No.: 19/095,074

(22) Filed: Mar. 31, 2025

(30) Foreign Application Priority Data
Feb. 3, 2025 (IN) 202511008672

(51) Int. Cl. G01N 33/02; B01L 3/00; G01N 27/327

(52) U.S. Cl. CPC G01N 33/02; B01L 3/502707; B01L 3/502753; G01N 27/3276; B01L 2300/0636; B01L 2300/0819; B01L 2300/123

(57) ABSTRACT

The present invention relates to a biosensor chip (100) for the detection of toxins in food and a method thereof. The biosensor chip (100) comprises a flexible substrate (102); a conductive layer (104) deposited on the flexible substrate (102); a bio-receptor (106) coated on the conductive layer (104) through a cross-linker (110); an analyzer (112) connected to the biosensor chip; and an operating device (114) to view the output. The length of the conductive layer (electrode) (104) is in range of 0.4 cm to 4 cm; angle between the two arms (116, 118) of the conductive layer (104) is in the range of 35°-65°; width of the conductive layer (104) is in range of 100 μm-1000 μm; and width between the two consecutive conductive layers (104) is in range of 100 μm-1000 μm, ensuring easy deployment of the biosensor chip (100) for rapid screening and quantification of toxins in food products.

### FIELD OF INVENTION

The present invention relates to a biosensor chip for the detection and analysis of toxins in food products and method for fabrication thereof. Particularly, the present invention relates to a portable biosensor chip with a flexible substrate, ensuring easy deployment of the device for rapid screening and quantification of toxins present in various food products and a method for fabrication thereof.

### BACKGROUND OF THE INVENTION

Numerous food commodities are prone to fungal infections. This contamination is because of the combination of environmental factors and improper agricultural practices such as incorrect harvesting in the field, transportation, or storage of crops. Especially in tropical regions, because of the hot and humid climate, improper ventilation, fungal invasion, and growth get accelerated. Therefore, the possibility of mycotoxins coexisting in the foods and derivatives thereof is very high. These contaminated commodities reach the humans or livestock directly by food consumption or indirectly after feeding the animals with potentially contaminated feedstuffs, which gets transferred to offspring during gestation or lactation. If feed and food contain exceptionally high levels of these mycotoxin compounds or low dosages but for a prolonged duration, they can cause adverse health effects, from acute to chronic Mycotoxicosis in both humans and animals due to the ingestion.

Biotoxins are toxic substances produced by animals, plants, and microorganisms. The majority of the biotoxins found in food are mycotoxins, which are secondary metabolites produced by fungi during the growth process and have toxic effects on human beings and animals. Ochratoxins, Fumonisins and Aflatoxins are common biotoxins and may enter the food chain through contaminated food and animal feed, causing serious health complications. Aflatoxin leads to DNA damage and prolonged exposure to aflatoxin cells accumulate DNA mutations thereby increasing the risk of developing liver cancer cells.

Aflatoxins are a family of mycotoxins, whose major metabolites are AFB1, AFB2, AFG1 and AFG2. Aflatoxin M1 (AFM1) is the main mono-hydroxylated form of AFB1. When mammals consume and ingest AFB1-contaminated feeds, it is metabolized and transformed in the liver, into AFM1 metabolites, which are further secreted through milk during lactation. Fumonisin B1 and Fumonisin B2 (FB1+FB2) mycotoxins are commonly produced by the fungus species Fusarium. These usually infect corn and other agricultural products like wheat flour, rice flour, oats flour. Ochratoxin A (OTA) is a naturally existing food-borne mycotoxin, that can be found in a diverse range of agricultural products around the world, including cereal grains, dried fruits, wine, and coffee. The WHO-IARC (International Agency for Research on Cancer) has investigated different mycotoxin compounds and has classified Aflatoxin M1 Group 1 (carcinogenic to humans), Fumonisin (FB1+FB2) and Ochratoxin A (OTA) as Group 2B (possibly carcinogenic to 2 humans), and 6]. Hence, to reduce end consumers' risks, mycotoxin-contaminated foods must be critically analyzed. Mycotoxins scientific evaluation of maximum residual/permissible limits (MRLs/MPLs), in various food and feed commodities have been established by various national and international regulations like the Food Safety and Standards Authority of India (FSSAI), the US Food and Drug Administration (FDA), European Union (EU) and Codex Alimentarius based on their harmful effects on people and animals.

Table 1 summaries the Maximum Permissible Levels of different mycotoxins in food and related products by different agencies:

| | | Maximum permissible level | | | | | | | |
|---|---|---|---|---|---|---|---|---|---|
| Mycotoxin | Product | FSSAI | | USFDA | | EU | | CODEX | |
| AFM1 | Milk and related | 0.5 | μg/kg | 0.5 | μg/kg | 0.05 | μg/kg | 0.5 | μg/kg |
| | products | (500 | ppt) | (500 | ppt) | (50 | ppt) | (500 | ppt) |
| FBs | Corn-derived | — | | 2000 | μg/kg | 200 | μg/kg | 200 | μg/kg |
| | foodstuffs | | | | | | | | |

| | | | | | | | | |
|---|---|---|---|---|---|---|---|---|
| OTA | Cereal grains | 20 | μg/kg | — | 5 | μg/kg | 5 | μg/kg |

| | | | | | | |
|---|---|---|---|---|---|---|
| Coffee-related | — | 5 | μg/kg | 5 | μg/kg | — |

products

There are several patent applications that relate to a device for detection and analysis of toxins in food products. One such Patent Application WO2014155391A2 discloses a device utilizes a sensing chip with a structured array of patterned electrodes for the detection and analysis of mycotoxins, including the integration of a biosensor with crosslinking elements and biosensing elements that can specifically bind to different mycotoxins. The manufacturing process involves creating a layered base, depositing conducting layers, and forming pits to expose electrodes for effective biosensor functionality enabling the device to perform simultaneous analysis of multiple mycotoxins in a single or multiple samples. However, the cited prior art employs rigid substrates such as glass or silicon wafers, which may limit the flexibility of the device. Further, the cited prior art discloses a static confinement of pits, which may not allow for real-time control over the flow of the fluid/particle within the microfluidic device. Furthermore, the cited prior art employs photolithography/thermal evaporation technique for fabricating the chip, which may be costly and a complex process.

Another Chinese Patent Application CN112986552A discloses a biochip for detecting aflatoxin B1 comprises a matrix chip, a composite film layer of 3-aminopropyltriethoxysilane and pyrrole, and a probe molecular layer, comprises at least one of an aldehyde group modified aptamer and an aflatoxin antibody; and the mass ratio of 3-aminopropyl triethoxysilane to pyrrole in the composite film layer of 3-aminopropyl triethoxysilane and pyrrole is 1:2-5. However, the cited prior art employs a composite film of APTES (aminopropyltriethoxysilane) and pyrrole, which may involve more complex synthesis and handling procedures, resulting in a higher cost of materials and processing steps. Further, the cited prior art uses a complex of aldehyde-modified aptamer and an aflatoxin B1 antibody together, which may require multiple chemical steps several chemical steps to modify the molecules and couple them together and thus may become more complex. Moreover, it may lead to stability, reproducibility issues as well as the potential for non-specific binding, which may minimize the sensitivity and specificity of the sensor. Furthermore, the use of two separate components i.e., aptamer (synthetic receptor) and antibody (natural bioreceptor) may result in a complex design of the biochip as well as increased production costs. Furthermore, the cited prior art employs steps using plasma activation, which may necessitate specialized equipment and careful control of different parameters of the process, which can make the process expensive and difficult to scale. Furthermore, the cited prior art employs the plasma coating method for depositing the conductive layer, which is a complex and expensive method and requires specialized equipment and controlled environments.

Furthermore, the above-cited prior arts fail to disclose biosensing devices having high robustness in harsh environmental conditions, flexibility, and low production cost.

In order to overcome the aforementioned problem associated with the state of the art, there exists a need to develop a portable and flexible biochip showing rapid response time with low concentration of analytes and high sensitivity, and capable of adapting to different food environments/products for the real-time and quick detection and analysis of toxins in food products and a method for fabrication thereof.

### OBJECTIVES OF THE INVENTION

The primary objective of the present invention is to provide a miniaturized, portable and flexible biosensor chip capable of adapting to different food environments/products for the detection of toxins in food and a method for fabrication thereof.

Another objective of the present invention is to provide a miniaturized, portable and flexible biosensor chip for the detection of toxins, particularly mycotoxins such as aflatoxin M1 in milk and milk products, AFM1 in paneer and whey protein; fumonisin B1 in wheat flour, rice flour, and oats flour; and ochratoxin A in wines, beer, coffee extracts.

Another objective of the present invention is to provide a portable and flexible biosensor chip requiring low concentrations of analytes for real-time and quick detection, and analysis and quantification of toxins in food products.

Another objective of the present invention is to provide a portable and flexible biosensor chip ensuring rapid detection and a short response time of less than 3 minutes.

Another objective of the present invention is to provide a portable and flexible biosensor chip showing high sensitivity, and thus capable of detecting even minute amounts of toxins.

Yet another objective of the present invention is to provide a rapid, low-cost, and economical method for fabricating the biosensor chip for the detection of toxins in food products.

Yet another objective of the present invention is to provide a fluidic channel integrated for the confinement of the sample fluid.

Yet another objective of the present invention is to provide a biosensor chip having a low detection limit and a large dynamic range of detection.

Other objectives and advantages of the present invention will become apparent from the following description taken in connection with the accompanying drawings, wherein, by way of illustration and example, the aspects of the present invention are disclosed.

### SUMMARY OF THE INVENTION

The present invention relates to a biosensor chip for the detection of toxins in food products. The biosensor chip comprises a flexible substrate for the fabrication of a biochip; a conductive layer deposited on the flexible substrate; a bio-receptor coated on the conductive layer through a cross-linker; an analyzer connected to the biochip; and an operating device equipped with a display, enabling the user to view the output provided by the analyzer. The length of the conductive layer (electrode) is in the range of 0.4 to 4 cm, and the angle between the two arms of the conductive layer (electrode) is in the range of 35-65°. The width of the conductive layer (electrode) is in the range of 100-1000 μm. The width between the conductive layers (electrode) is in the range of 100-1000 μm, ensuring easy deployment of the biosensor chip for rapid screening and quantification of toxins in food products. The present invention also provides a method for the fabrication of the biosensor chip. The biosensor chip fabricated in the present invention is a portable and flexible chip requiring low concentrations of analytes for real-time and quick detection analysis and quantification of toxins in food products.

### BRIEF DESCRIPTION OF DRAWINGS

The present invention will be better understood after reading the following detailed description of the presently preferred aspects thereof with reference to the appended drawings, in which the features, other aspects, and advantages of certain exemplary embodiments of the invention will be more apparent from the accompanying drawing.

FIG. 1(a) illustrates a front view of an inverted V-shaped flexible biosensor chip, and FIG. 1(b) shows an isometric view of the inverted V-shaped flexible biosensor chip;

FIG. 2 illustrates an arrangement of the layers for fabricating a biosensor chip;

FIG. 3 illustrates a schematic representation of the biosensing of food toxins using the flexible biosensor chip;

FIG. 4(a) illustrates the FESEM image of a bare flexible biosensor chip; and FIG. 4(b) illustrates the FESEM image of the flexible biosensor chip coupled with bio-receptors;

FIG. 5(a) illustrates a probe station of connection of a bent-shaped flexible biosensor chip; and FIG. 5(b) illustrates graphical representation depicting the response of the inverted-V shaped flexible biosensor chip in sensing of 500 ppt AFM1 spiked in paneer slurry and blank;

FIG. 6 illustrates a graphical representation depicting the response of the inverted-V-shaped biosensor chip obtained from sensing of AFM1 spiked in whey protein over the non-spiked sample (blank);

FIG. 7(a) illustrates a probe station connection of the inverted-V shaped flexible biosensor chip; and FIG. 7(b) illustrates graphical representation depicting the response of the inverted-V shaped biosensor chip in sensing of 500 ppt AFM1 spiked in milk from blank;

FIG. 8 illustrates a graphical representation depicting a linear calibration curve obtained for sensing different concentrations of AFM1 in milk, R²=0.9911(n=3); and

FIG. 9 illustrates a flexible biosensor chip showing the fluid confinement in the channel along with the connecting points.

### DETAILED DESCRIPTION OF THE INVENTION

The following description describes various features and functions of the disclosed system. The illustrative aspects described herein are not meant to be limiting. It may be readily understood that certain aspects of the disclosed system can be arranged and combined in a wide variety of different configurations, all of which have not been contemplated herein.

Accordingly, those of ordinary skill in the art will recognize that various changes and modifications of the embodiments described herein can be made without departing from the scope of the invention. In addition, descriptions of well-known functions and constructions are omitted for clarity and conciseness.

Features that are described and/or illustrated with respect to one embodiment may be used in the same way or in a similar way in one or more other embodiments and/or in combination with or instead of the features of the other embodiments.

The terms and words used in the following description are not limited to the bibliographical meanings but are merely used to enable a clear and consistent understanding of the invention. Accordingly, it should be apparent to those skilled in the art that the following description of exemplary embodiments of the present invention are provided for illustrative purposes only and not for the purpose of limiting the invention.

It is to be understood that the singular forms “a”, “an” and “the” include plural referents unless the context clearly dictates otherwise.

It should be emphasized that the term “comprises/comprising” when used in this specification is taken to specify the presence of stated features, steps or components but does not preclude the presence or addition of one or more other features, steps, components or groups thereof. The equations used in the specification are only for computation purposes.

The term “biosensor chip” may be interchangeably used with the term “biochip” in the present disclosure.

Accordingly, the present invention relates to a biosensor chip for the detection and analysis of toxins in food products and a method for fabrication thereof. Particularly, the present invention relates to a miniaturized, portable inverted V-shaped biosensor chip with a flexible substrate, ensuring easy deployment of the device for rapid screening and quantification of toxins present in various food products and a low-cost easy to perform at scale method for fabrication thereof. The biosensor chip fabricated in the present invention is effective in the detection of toxins in more complex matrices such as paneer (Indian cottage cheese) and whey protein both flavored and non-flavored.

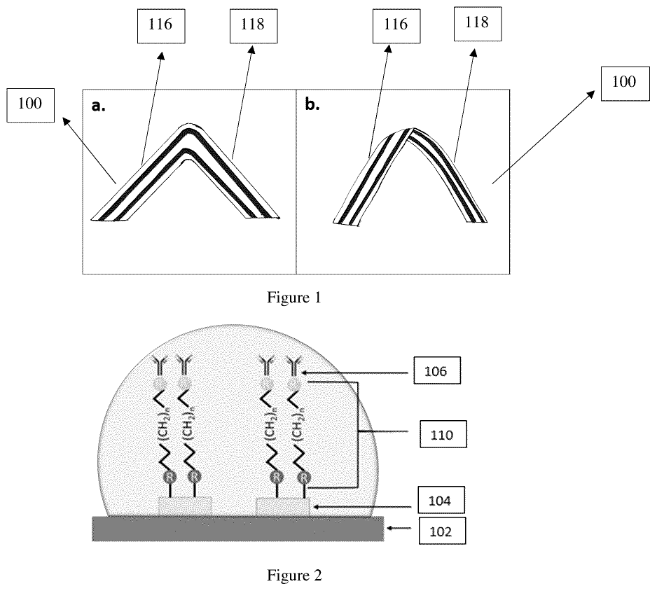

In an embodiment, as shown in FIG. 1-3, the biosensor chip (100) for the detection of toxins in food comprises the following components:
(a) A flexible substrate (102): The flexible substrate (102) is configured to provide structural support for the fabrication of the biosensor chip (100). The flexible substrate (102) imparts flexibility to the biosensor chip (100), enabling the biosensor chip (100) to conform to irregularly shaped food samples having different surface properties, and thereby maintaining direct contact with the food samples, which in turn enhances the accuracy of detection of toxin. In an exemplary embodiment, the flexible substrate (102) is prepared from a material selected from the group consisting of, such as, but not limited to polyethylene terephthalate (PET) sheet, kapton sheet, thermoplastic polyamine (TPA) sheet, and textiles and cellulose fibers.
(b) A layer of conductive material (104): The layer of conductive material (104) is deposited on the flexible substrate (102). The conductive ink forms an electrical path for the transmission of signals in the biosensor chip (100), thereby enabling real-time detection of biomolecular interactions. As shown in FIG. 9, the conductive layer also creates a channel (120) for the confinement of the fluid. In an exemplary embodiment, the conductive material may be selected from a group consisting of, such as, but not limited to gold, copper, silver, carbon, aluminum, or a combination thereof.
(c) A bio-receptor (106): The bio-receptor (106) is coated on the conductive material-coated flexible substrate through a cross-linker (110) to form a biosensor chip (100). The bio-receptor (106) is a biological molecule that specifically binds with the toxins present in food products. In an exemplary embodiment, bio-receptor (106) may be selected from a group consisting of, such as, but not limited to, antibodies (monoclonal and polyclonal), aptamers, DNA, whole cells, and the like.
(d) A cross-linker (110): The cross-linkers (110) help to bind the bio-receptor (106) to the conductive material-coated flexible substrate characterized by Field Emission Scanning Electron Microscopy (FE-SEM) (as shown in FIG. 4). In an exemplary embodiment, the cross-linker (110) has the chemical formula R—X—R′ wherein R may be selected from a head group consisting of, such as, but not limited to thiol (—SH), primary amine (—NH₂), methionine (NH₂—CH—COOH), and phosphate (PO₄); X may be a carbon chain moiety selected from the range of C3 to C18; and R′ end group may be selected from the group consisting of, such as, but not limited to amine, carboxyl, carboxylic acid, and cyanide groups.

In an exemplary embodiment, the cross-linker (110) used in the present invention may be selected from the group consisting of, such as, but not limited to, cysteine hydrochloride, cysteamine hydrochloride, arginine, glutamic acid, 3, 3′-dithiodipropionic acid, 3-mercaptopropionic acid, 3, 3′-dithiodipropionic acid di(N-hydroxy succinimide ester), 4,4′-dithiodibutyric acid, 11-amino-1-undecanethiol, 11-mercaptoundecanoic acid, 11-phosphonoundecanoicacid, and 16-phosphonohexadecanoic acid, or a combination thereof.
(e) An analyzer (112): The analyzer (112) is connected to the biosensor chip (100) through wired or wireless communication means to interpret and process the electrical signals generated by the bioreceptor to the toxins present in the food and generate an output indicating the presence and concentration of specific toxins in the food. In an exemplary embodiment, the analyzer (112) used in the present invention may be an electrochemical analyzer.
f) An operating device (114): The operating device (114) enables the user to view the output generated by the analyzer (112) on the display of the operating device (114). In an exemplary embodiment, the operating device may be selected from a group consisting of, such as, but not limited to, mobile devices, laptops, or any computing device.

In an exemplary embodiment, the analyzer may be a standalone device or part of an operating device.

The biosensor chip (100) fabricated in the present invention is used to detect different toxins present in different food products such as, but not limited to, aflatoxin M1 in milk, milk powder, paneer, whey protein, cheese, khova; fumonisin B1 (FB1) present in wheat flour, rice flour, oats flour; and ochratoxin A (OTA) present in beverages like wines, beer, malt, coffee beans and extracts, etc.

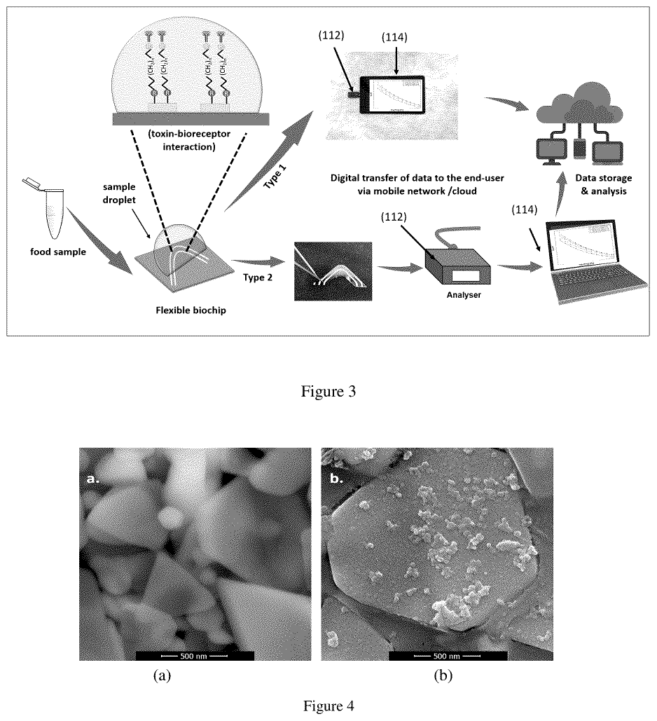

In an embodiment, as shown in FIG. 3, when the bioreceptor on the biosensor chip interacts with toxins present in the food product, the conductive layer helps in converting these interactions into measurable electrical signals, which may further be analyzed by the analyzer to detect the specific type of the toxin in the food sample and concentration of the detected toxin.

The biosensor chip (100) fabricated on the flexible substrate (102) is designed with specific geometry including shape and dimension, for the detection and analysis of food toxins. The biosensor chip (100) may be fabricated on various kinds of flexible substrates (102) so as to adapt to different food environments such that the biosensor chip can conform to irregularly shaped food samples having different surface properties, thereby maintaining direct contact with the food samples, which in turn enhances the accuracy of detection of toxin.

In an embodiment, the conductive layer (104) deposited on the flexible substrate is coated with a specific bio-receptor (106) layer that constitutes the biosensor chip, which is used for detecting toxins present in food products. In an exemplary embodiment, the conductive layer (104) may be deposited using an inkjet printing technique and heat curing technique. The conductive ink may be dispensed using a smart dispenser integrated with the inkjet printing technique. The conductive ink is placed in a cartridge attached to a smart dispenser, which may be such as, but not limited to, a mechanical robotic arm. In another exemplary embodiment, the nozzle diameter used to dispense the ink on the flexible substrate may be in a range of 100-225 μm, allowing highly accurate printing of conductive traces on flexible substrates (102), thereby ensuring fine resolution and consistent quality. The smart dispenser traces the conductive layer (electrode) (104) path on the flexible substrate (102). After printing, the inkjet-printed electrodes may be heat-cured at a temperature ranging between 100-150° C. for 10-15 minutes. The time duration and temperature of heat-curing depend on the substrate and the conductive ink used. To heat cure, the electrodes may be kept on the temperature and time-regulated hot plate. For instance, when Silver conductive ink is inkjet printed on a PET flexible substrate, the heat curing temperature and time may be optimized to 120° C. for 15 minutes, respectively.

In a preferred embodiment, the biosensor chip was designed in form of an inverted V-shape having a specific geometry wherein the length of the conductive layer (electrode) (104) ranges from 0.4 to 4 cm; the angle between the two arms (116, 118) of the conductive layer (electrode) (104) (as shown in FIG. 1a and FIG. 1b) is in the range of 35-65°; the width of the conductive layer (electrodes) (104) is in range of 100-1000 μm; and the width between the two consecutive conductive layers (electrode) (104) is in range of 100-1000 μm. The length of the conductive layer (electrode) (104) and the width of the conductive layer (electrode) (104) is required to be in the range of 0.4 to 4 cm and 100-1000 μm, respectively as it enables the flexible electrodes to be miniaturized electrodes, thus easy to deploy/mount on any food environment. The length of the conductive layer (electrode) (104) and the width of the conductive layer (electrode) (104) are directly proportional to the area of the conductive ink. The length of the conductive layer (electrode) (104) and width of the conductive layer (electrode) enable flexible electrodes to maintain both the features of miniaturization and capacitive nature. Further, as the capacitance response parameter of the flexible electrode is considered for analysis, the width between two consecutive conductive layers (electrodes) (104) is required to be in a range of 100-1000 μm. The width or the distance between the conductive layers (electrodes) is inversely proportional to the capacitance parameter of the flexible biosensor chip (100). Further, since the arms of the inverted-V-shaped flexible biosensor chip (100) act as the channel for fluid confinement, an angle between two arms (116, 118) of the conductive layer (electrode) (104) is required to be in a range of 35-65°. This feature also allows the incorporation of the microfluidic handling of the samples. The unique combination of the dimensions of the flexible biosensor chip (100), such as length, width, angle, and confinement volume, enables us to achieve the desired detection limit and linear range for quantitative measurement of toxins.

In an embodiment, FIG. 9 shows fluid confinement in the channel (120) along with the connecting points (1,2 and 3,4). The printed conductive ink on the flexible substrate (102) acts as a channel to confine the sample fluid drop-casted on it for further analysis. Further, the connections to the analyzer (112) may be made from either side, i.e., 1 and 2 or 3 and 4 points, making the flexible biosensor chip (100) capable of being deployed in different food environments.

In an embodiment, the present invention relates to a method for the fabrication of the biosensor chip for the detection of toxins in food products. The method comprises the following steps:
(a) preparing buffer solutions with pH ranges suitable for biological applications;
(b) preparing analyte stock solutions in appropriate solvents;
(c) preparing complementary antibody dilutions;
(d) preparing reference materials for the selected food matrices.
(e) selecting a flexible substrate (102) for the fabrication of a biosensor chip (100);
(f) depositing a conductive layer (electrode) (104) on the flexible substrate (102);
(g) defining the geometry of the conductive layer (104) wherein the length of the conductive layer (electrode) ranges from 0.4 cm to 4 cm; the width of the conductive layer (electrodes) ranges from 100 to 1000 μm; the width between two consecutive conductive layers (electrodes) ranges from 100 to 1000 μm; and the angle between two arms of the conductive layer (electrode) (104) is in a range of 35 to 65 degrees;
(h) curing the deposited conductive layer (104);
(i) washing the conductive layer (104);
(j) drying the wet conductive layer (104);
(k) forming a cross-linking self-assembled monolayer on the conductive layer (104);
(l) activating end groups of the cross-linking self-assembled monolayers;
(m) immobilizing bio-receptors (106) on the activated flexible substrate (102) through the cross-linkers (110), resulting in a flexible biosensor chip (100);
(n) connecting the flexible biosensor chip (100) to an analyzer (112);
(o) connecting the analyzer (112) to an operating device (114);
(p) applying an alternating current perturbation to the flexible biosensor chip (100);
(q) sweeping frequency across a predetermined range;
(r) comparing responses between blank and spiked samples for further analysis; and
(s) viewing the output provided by the analyzer (112) on the operating device (114).

In an exemplary embodiment, the buffer solutions may be selected from a group consisting of, such as, but not limited to, phosphate-buffered saline (PBS) with a pH range of 7.0-7.6; and carbonate buffer with a pH range of 9.0-10.4.

In another exemplary embodiment, the analytes may be selected from a group consisting of, such as, but not limited to, AFM1, FB1, and OTA.

In another exemplary embodiment, the conductive layer is deposited using inkjet printing technology, wherein a conductive ink cartridge is placed in a smart dispenser utilizing a nozzle with a diameter ranging from 100-225 μm to deposit the conductive layer on the flexible substrate with precision.

In another exemplary embodiment, the curing process involves heat curing at temperatures between 100-130° C. for 10-15 minutes. In an embodiment, the heat curing time duration and temperature depend on the substrate and the conductive ink used.

In another exemplary embodiment, the conductive layer (104) may be washed with deionized water.

In another exemplary embodiment, the wet conductive layer (104) may be dried under ultrapure nitrogen (99.95).

In yet another exemplary embodiment, the cross-linking self-assembled monolayer is formed using a concentration range of 1-50 mM over a period of 1-20 hours.

In yet another exemplary embodiment, the activation of end groups may be performed over a duration of 0.5-2 hours.

In yet another exemplary embodiment, the AC perturbation may be applied in the range of 5-20 mV, with frequency sweeping between 0.1 Hz-100 kHz.

The following illustrates an example of the method for the fabrication of a biosensor chip for the detection of toxins in food products and should not be construed to limit the scope of the invention. In an exemplary embodiment, the method for fabricating a biosensor chip in the present invention comprises the following steps:

### 1) Preparation of Buffer Solution:

(a) Preparation of Phosphate Buffer Saline: Sodium dihydrogen phosphate (monohydrate) (NaH₂PO₄·H₂O) and disodium hydrogen phosphate was dissolved separately in DI water to prepare 100 mM of two solutions respectively. 100 mM of Na₂HPO₄ solution was added to 100 mM NaH₂PO₄·H₂O until a desired pH in the range of 7.0-7.6 pH is reached. To this, 50 mM of Sodium Chloride (NaCl) solution was added to prepare PBS.
(b) Preparation of Carbonate Buffer solution: Carbonate buffer solution is used as the coating buffer for antibody immobilization. Sodium bicarbonate (NaHCO₃) and Sodium carbonate (Na₂CO₃) were dissolved separately in DI water to prepare 50 mM of two solutions. 50 mM of NaHCO₃ is added to Na₂CO₃, till a desired pH in the range of 9.0-10.4 pH is reached. It's stored at 4° C.
(c) Preparation of AFM1 stock solution: The AFM1 stock solution was prepared as follows: 5 g AFM1 powder was dissolved in 1 mL 100% ACN and stored at 4° C. Working standards from this are prepared by diluting 5 g/mL AFM1 concentration with 5% ACN (v/v) in PBS
(d) Preparation of AFM1 antibody solutions
   The AFM1 monoclonal antibodies were procured commercially. The 100 g (1 mg/mL) of antibodies were diluted suitably in pyrogen-free, millipore deionized water to obtain the standard solution of AFM1 antibodies. These are then split into aliquots and stored at −18° C. to prevent the freeze-thaw cycle, which will subsequently reduce the activity of the antibodies. From these aliquots, further dilutions of antibodies are prepared in a carbonate coating buffer.
(e) Preparation of ERM-BD (zero level-AFM1) milk samples
   The European Reference Materials-BD-282 (ERM) zero-level AFM1 was procured from Joint Research Centre (JRC), EU. From that, ERM-BD-282 powder was dissolved in warm PBS. It is then centrifuged between 4000-8000 rpm for 10-15 minutes. The upper-fat layer was removed, and the middle portion was used for further analysis.

### 2) Functionalization of the Flexible Biosensor Chip:

(a) Inkjet printing of conductive layer (electrode) (104) on the flexible substrate for fabrication of the biosensor chip (100): The conductive ink is attached to the smart dispenser. To inkjet print, the nozzle diameter used to dispense the ink on the substrate ranges from (100-225 μm). It is then inkjet printed on the flexible substrate. The dimensions of the flexible biosensor chip (100) are as follows:
   the length of the conductive layer (electrode) (104) ranges from 0.4 cm to 2 cm
   the angle between two arms of the conductive layer (electrode) (104) is in a range of 35-65°
   the width of the conductive layer (electrode) (104) ranging from 100-900 μm,
   the width between the two consecutive conductive layers (electrodes) (104) ranges from 100-1000 μm.
(b) Heat curing of the electrode: The inkjet-printed electrodes were heat-cured at temperatures above 100-130° C. for 10-15 minutes. The heat curing time duration and temperature depend on the substrate and the conductive ink used.
(3) Cleaning of flexible biosensor chip (100): The electrodes were washed with DI water, followed by drying under ultrapure nitrogen (99.9%).
(4) Formation of cross-linking self-assembled monolayer (Thiolation): 2-8 mM ethanolic solution of 11-MUA is prepared using absolute ethanol. This solution is drop-casted on the flexible electrodes. The volume used for drop-casting is dependent on the dimensions of these flexible electrodes. After drop-casting, the solution is kept for 12-16 hours at room temperature in a moist environment. It is then removed and washed thoroughly with DI water and absolute ethanol and is N₂ purged.
(5) Activation of the crosslinking layer: Equimolar concentration of EDC/NHS is prepared using DI water. This is used to activate the —COOH end of the crosslinked flexible electrodes. It was then drop-casted and kept for 1-2 hours at room temperature. Then, the electrodes are washed with DI water to remove unbound excess EDC/NHS.
(6) Immobilization of bio-receptors on the flexible biosensor chip (100): After activation of the flexible biosensor chip (100), antibody dilution was drop-casted on the electrode over the activated surface and kept overnight at 2-6° C. Then it was washed with DI water to remove the unbounded antibodies and N₂ dried.
(7) Measurement parameters: The AC perturbation potential in the range of 5-20 mV was applied over a frequency range of 1 Hz-10 kHz. For the measurements, electrodes were connected to the impedance analyzer, through a two-point connector. For sensing, the response curves between the blank ERM-BD 282 and the AFM1 spiked ERM-BD 282 are compared and used for further analysis.

The following illustrates experimental data for the detection of AFM1 in different food samples using the biosensor chip (100) fabricated in the present invention and should not be construed to limit the scope of the invention.

### Experimental Data

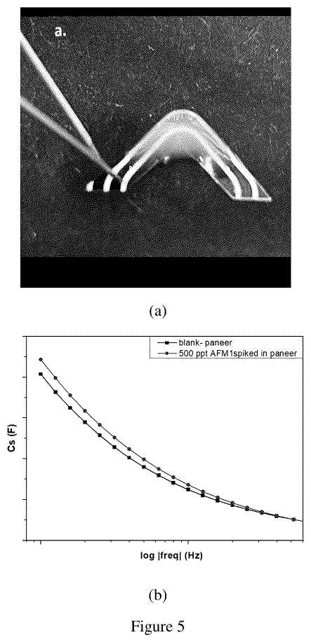

Bio-sensing of AFM1 spiked in commercial paneer (Indian Cottage cheese) samples on the flexible biosensor chip: Paneer (Indian Cottage cheese) sample preparation: Commercially available paneer was procured off the shelf from the local market and was made into a slurry using warm PBS (70° C.) followed by sonication for 5 minutes. It was divided into two portions i.e., the blank (slurry itself) and 500 ppt AFM1 spiked in the slurry. In this case, the biochip itself was dipped into the blank paneer slurry for 30 seconds. For continuity, PBS is then added to the biochip, and a response is taken. The response in black (filled squares) is for the blank paneer slurry and the response in red (filled circles) is for the spiked paneer slurry with 5 minutes of incubation of the biochip in the slurry sample. The responses obtained for blank and spiked paneer samples are presented in FIG. 5(b). 500 ppt of AFM1 in the paneer sample was successfully detected using the biochip. FIG. 5 (a) shows the connection of the flexible biosensor chip to the probe station, which in turn is connected to the analyzer (112) to obtain the sensing response.

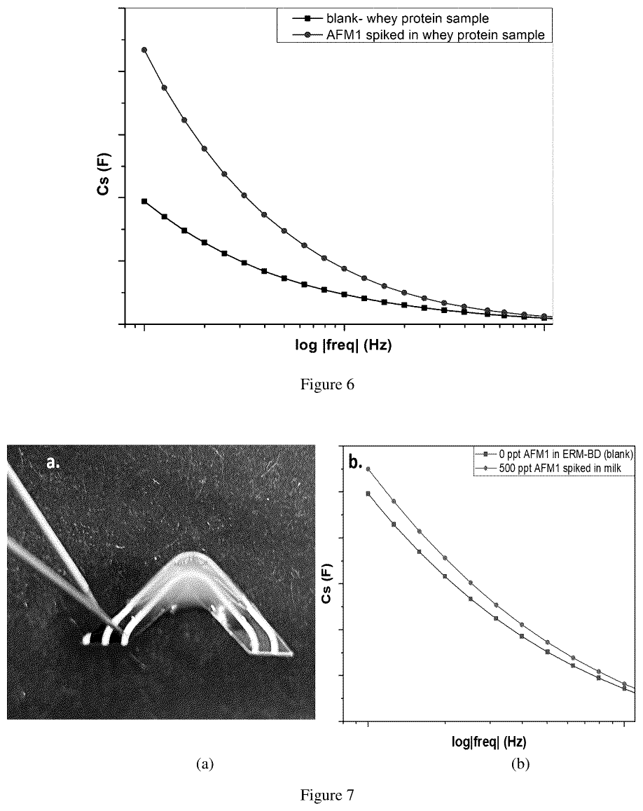

Bio-sensing of AFM1 in commercial whey protein samples using the flexible biosensor chip: Whey protein sample preparation: Commercially available whey protein was procured off the shelf from the local market. It was reconstituted with warm phosphate buffer saline (PBS) at a temperature of 60-70° C., followed by centrifugation of the sample for 10 minutes at 4000-8000 rpm. After centrifugation, the whey protein and the other undissolved matrix, if any, get separated out. The middle layer of the whey protein sample, which is fat-free, is extracted for analysis of toxins. The whey protein thus collected was divided into two portions. The first portion is designated as blank, without externally added AFM1, and AFM1 in the range 20-1000 ppt AFM1 were spiked in the second portion of the whey protein sample. About 10-50 μL of whey protein sample was drop-casted on the bio-chip and was analyzed electrochemically. As shown in FIG. 6, the response signal (represented sampling points as filled black squares) was for the blank whey protein sample, and the response signal (represented sampling points as filled red circles) represented the spiked whey protein sample with 5 minutes of incubation of biochip. It is evident clearly that AFM1 was easily measurable in the spiked samples over the blank.

Bio-sensing of AFM1 spiked in ERM-BD milk samples on the flexible biosensor chip: Milk sample preparation: The milk powder reference material ERM-BD 282 is reconstituted with warm phosphate buffer saline (PBS) followed by centrifugation of the sample for 10 minutes. After centrifugation, the upper-fat layer is removed, and the middle layer of the milk, which is fat-free, is extracted for analysis of toxins.

The food toxin sample gets exposed to the biosensor chip (100). Measurements of the concentrations of toxins were performed under the optimized conditions of applied potential and frequency. FIG. 7(a) shows the connection of the flexible biosensor chip to the probe station, which in turn is connected to the analyzer (112) to obtain the sensing response. FIG. 7(b) shows the response signal obtained during the sensing of AFM1 spiked in milk samples using the biosensor chip. About 10-50 μL of milk sample is drop-casted on the biochip. The response curve in black (filled squares) is for the blank sample, which is the European Reference Material, ERM-BD 282, certified to have 0 ppt AFM1. It is followed by the response curve in red (filled circles) for 500 ppt AFM1 spiked in ERM-BD milk samples. The difference between the responses of the two samples confirms the presence of AFM1.

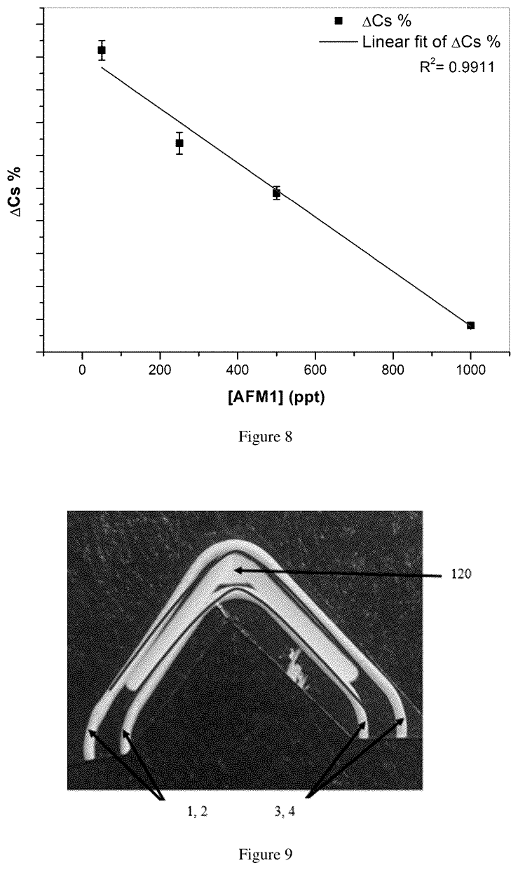

The calibration curve for biosensing of AFM1 spiked in ERM-BD milk samples on the flexible biosensor chip: Electrical responses of 50, 250, 500, and 1000 ppt AFM1 spiked in ERM-BD milk samples were obtained. The responses obtained after a minimal incubation time of 10 minutes at room temperature for different concentrations of AFM1 in milk (n=3) are quite distinguishable, as represented in the calibration curve in FIG. 8. The flexible biochip is successfully able to detect AFM1 as low as 50 ppt AFM1 in milk (EU limits for AFM1 in milk) with a wide linear range of 50 ppt-1000 ppt AFM1 spiked in milk samples. This also complies the USFDA and FSSAI Indian regulatory limits of 500 ppt AFM1 in milk samples.

The advantages of the present invention are discussed herein:
The present invention is to provide a flexible biosensor chip for the detection of toxins in different food products/environments.
The present invention is to provide a fluidic channel integrated for the confinement of the sample fluid.
The present invention provides the method for the detection of aflatoxin M1 in milk and milk products such as paneer, whey protein, cheese, khova, etc.
The present invention is to provide a method and flexible biosensor chip (device) capable of rapidly screening and quantifying toxins present in food products.
The present invention is to provide a rapid, low-cost cost, and economical method for the fabrication of the biosensor chip for the detection of toxins in food products.
The present invention is to provide a biosensor chip having a low detection limit and a large dynamic range of detection.
The present invention is to provide the biosensors that emerged as important analytical instruments for addressing the complexities of agricultural and food safety applications because they have shown great promise in various applications, including medical diagnostics and environmental monitoring.
The present invention is to provide a biosensor chip, utilizing the precision and discrimination of biological recognition components enabling the rapid detection of target analytes with minimal sample preparation.
The present invention is to provide real-time monitoring, multiplexed detection, and compatibility with various sample matrices.

While this invention has been described in connection with what is presently considered to be the most practical and preferred embodiment, it is to be understood that the invention is not limited to the disclosed embodiments, but, on the contrary, is intended to cover various modifications and equivalent arrangements included within the scope of the appended claims.

We claim:

1. A biosensor chip (100) for the detection of toxins in food products, comprising:
a. a flexible substrate (102) for the fabrication of a biosensor chip;
b. a layer of conductive material (104) deposited on the flexible substrate (102) to form an electrical path for transmission of signals in the biosensor chip (100) and create a channel (120) for the confinement of the fluid;
c. a bio-receptor (106) coated on the conductive material-coated flexible substrate through a cross-linker (110) to form a biosensor chip (100); and
d. an analyzer (112) connected to the biosensor chip through a wired or wireless communication means to interpret and process the electrical signals generated by the bioreceptor binding to the toxins present in the food and generate an output indicating the presence and concentration of specific toxins in the food; and
e. an operating device (114) equipped with a display to view the output generated by the analyzer (112);
wherein,
the length of the conductive layer (electrode) ranges from 0.4 cm to 4 cm;
the width of the conductive layer (electrodes) ranges from 100 μm to 1000 μm;
the width between two consecutive conductive layers (electrodes) ranges from 100 μm to 1000 μm; and
the angle between two arms of the conductive layer (electrode) (104) is in a range of 35 degrees to 65 degrees.

2. The biosensor chip (100) for the detection of toxins in food products as claimed in claim 1, wherein the flexible substrate (102) is prepared from a material selected from the group consisting of polyethylene terephthalate (PET) sheet, kapton sheet, thermoplastic polyamine (TPA) sheet, and textiles and cellulose fibers.

3. The biosensor chip (100) for the detection of toxins in food products as claimed in claim 1, wherein the conductive material (104) is selected from the group consisting of gold, copper, silver, carbon, aluminum, or a combination thereof.

4. The biosensor chip (100) for the detection of toxins in food products as claimed in claim 1, wherein the bio-receptor (106) is selected from a group consisting of monoclonal antibodies. polyclonal antibodies, aptamers, DNA, whole cells, and the like.

5. The biosensor chip (100) for the detection of toxins in food products as claimed in claim 1, wherein the cross-linker (110) has the chemical formula R—X—R′ wherein
R is selected from a head group consisting of thiol (—SH), primary amine (—NH₂), methionine (NH₂—CH—COOH), and phosphate (PO₄);
X is a carbon chain moiety selected from the range of C3 to C18; and
R′ end group is selected from the group amine, carboxyl, carboxylic acid, and cyanide groups.

6. The biosensor chip (100) for the detection of toxins in food products as claimed in claim 1, wherein the cross-linker (110) is selected from the group consisting of cysteine hydrochloride, cysteamine hydrochloride, arginine, glutamic acid, 3, 3′-dithiodipropionic acid, 3-mercaptopropionic acid, 3, 3′-dithiodipropionic acid di(N-hydroxysuccinimide ester), 4,4′-dithiodibutyric acid, 11-amino-1-undecanethiol, 11-mercaptoundecanoic acid, 11-phosphonoundecanoic acid, and 16-phosphonohexadecanoic acid, or a combination thereof.

7. The biosensor chip (100) for the detection of toxins in food products as claimed in claim 1, wherein the analyzer (112) is a standalone device or part of an operating device (114).

8. The biosensor chip (100) for the detection of toxins in food products as claimed in claim 1, wherein the analyzer (112) is an electrochemical analyzer.

9. The biosensor chip (100) for the detection of toxins in food products as claimed in claim 1, wherein the operating device (114) is selected from a group consisting of mobile devices, laptops, or any computing device.

10. A method for fabricating the biosensor chip (100) as claimed in claim 1, comprising steps of:
(a) preparing buffer solutions with pH ranges suitable for biological applications;
(b) preparing analyte stock solutions in appropriate solvents;
(c) preparing complementary antibody dilutions;
(d) Preparing reference materials for the selected food matrices.
(e) selecting a flexible substrate (102) for the fabrication of a biosensor chip (100);
(f) depositing a conductive layer (electrode) (104) on the flexible substrate (102);
(g) defining the geometry of the conductive layer (104) wherein the length of the conductive layer (electrode) ranges from 0.4 cm to 4 cm; the width of the conductive layer (electrodes) ranges from 100 to 1000 μm; the width between two consecutive conductive layers (electrodes) ranges from 100 to 1000 μm; and the angle between two arms of the conductive layer (electrode) (104) is in a range of 35 to 65 degrees;
(h) curing the deposited conductive layer (104);
(i) washing the conductive layer (104);
(j) drying the wet conductive layer (104);
(k) forming a cross-linking self-assembled monolayer on the conductive layer (104);
(l) activating end groups of the cross-linking self-assembled monolayers;
(m) Immobilizing bio-receptors (106) on the activated flexible substrate (102) through the cross-linkers (110), resulting in a flexible biosensor chip (100);
(n) connecting the flexible biosensor chip (100) to an analyzer (112);
(o) connecting the analyzer (112) to an operating device (114);
(p) applying an alternating current perturbation to the flexible biosensor chip (100);
(q) sweeping frequency across a predetermined range;
(r) comparing responses between blank and spiked samples for further analysis; and
(s) viewing the output provided by the analyzer (112) on the operating device (114).

11. The method for fabricating the biosensor chip (100) as claimed in claim 10, wherein the buffer solutions are selected from a group consisting of phosphate-buffered saline (PBS) with a pH range of 7.0-7.6, and carbonate buffer with a pH range of 9.0-10.4.

12. The method for fabricating the biosensor chip (100) as claimed in claim 10, wherein the analytes are selected from a group consisting of AFM1, FB1, and OTA.

13. The method for fabricating the biosensor chip (100) as claimed in claim 10, wherein the conductive layer (104) is deposited using inkjet printing technology wherein a conductive ink cartridge is placed in a smart dispenser utilizing a nozzle with a diameter ranging from 100-225 μm to deposit the conductive layer (104) on the flexible substrate (102) with precision.

14. The method for fabricating the biosensor chip (100) as claimed in claim 10, wherein the curing process involves heat curing at temperatures between 100-130° C. for 10-15 minutes.

15. The method for fabricating the biosensor chip (100) as claimed in claim 10, wherein the conductive layer (104) is washed with deionized water.

16. The method for fabricating the biosensor chip (100) as claimed in claim 10, wherein the wet conductive layer (104) is dried under ultrapure nitrogen.

17. The method for fabricating the biosensor chip (100) as claimed in claim 10, wherein the cross-linking self-assembled monolayer is formed using a concentration range of 1-50 mM over a period of 1-20 hours.

18. The method for fabricating the biosensor chip (100) as claimed in claim 10, wherein the activation of end groups is performed over a duration of 0.5-2 hours.

19. The method for fabricating the biosensor chip (100) as claimed in claim 10, wherein the AC perturbation may be applied in the range of 5 mV-20 mV, with frequency sweeping between 0.1 Hz-100 kHz.
